(19) United States
(12) Patent Application Publication
(10) Pub. No.: US 2026/0120274 A1
(43) Pub. Date: Apr. 30, 2026

(54) METHODS AND APPARATUS FOR HISTO-PROJECTION BASED IMAGE RECONSTRUCTION USING DEEP LEARNING PROCESSES

(71) Applicants: Siemens Medical Solutions USA, Inc., Malvern, PA (US); The Trustees of The University of Pennsylvania, Philadelphia, PA (US)

(72) Inventors: Vladimir Panin, Knoxville, TN (US); Mael Millardet, Knoxville, TN (US); Deepak Bharkhada, Knoxville, TN (US)

(21) Appl. No.: 19/058,641

(22) Filed: Feb. 20, 2025

Related U.S. Application Data
(60) Provisional application No. 63/711,878, filed on Oct. 25, 2024.

(51) Int. Cl. G06T 7/00; G06T 5/40; G16H 50/20

(52) U.S. Cl. CPC G06T 7/0012; G06T 5/40; G16H 50/20; G06T 2207/10104; G06T 2207/20081; G06T 2207/20084

(57) ABSTRACT

Systems and methods for training machine learning processes based on histo-projections, and for reconstructing medical images based on the trained machine learning processes, are disclosed. In some examples, a computing device receives image measurement data from an image scanning system, such as a positron emission tomography (PET) imaging system. The computing device applies a histogramming process to the image projection data and, based on applying the histogramming process, generates histo-projection data. Further, the computing device applies a trained machine learning process to the histo-projection data and, based applying the trained machine learning process to the histo-projection data, generates a reconstructed image. The computing device may provide the reconstructed image for display.

### RELATED APPLICATIONS

This application claims the benefit of priority to U.S. Application No. 63/711,878, entitled “Deep Learning Image Reconstruction From Histo-Projections,” and filed on Oct. 25, 2024, the entire disclosure of which is expressly incorporated herein by reference.

### FIELD

Aspects of the present disclosure relate in general to medical diagnostic systems and, more particularly, to reconstructing images from nuclear imaging systems for diagnostic and reporting purposes.

### BACKGROUND

Nuclear imaging systems can employ various technologies to capture images. For example, some nuclear imaging systems employ positron emission tomography (PET) to capture images. PET is a nuclear medicine imaging technique that produces tomographic images representing the distribution of positron emitting isotopes within a body. Some nuclear imaging systems employ computed tomography (CT), for example, as a co-modality. CT is an imaging technique that uses x-rays to produce anatomical images. Magnetic Resonance Imaging (MRI) is an imaging technique that uses magnetic fields and radio waves to generate anatomical and functional images. Some nuclear imaging systems combine images from PET and CT scanners during an image fusion process to produce images that show information from both a PET scan and a CT scan (e.g., PET/CT systems). Similarly, some nuclear imaging systems combine images from PET and MRI scanners to produce images that show information from both a PET scan and an MRI scan.

In at least some cases, the nuclear imaging systems capture measurement data, and process the captured measurement data using mathematical algorithms to reconstruct medical images. For example, reconstruction can be based on machine learning models, such as machine learning models based on deep learning algorithms. Typically, the machine learning models are trained and once trained to a target degree, are employed in practice to diagnose patients. Even after robust training, however, the machine learning models can maintain algorithmic biases that lead to errors (e.g., hallucinations) within reconstructed images. Moreover, these reconstruction processes can provide blurry images, thereby causing impediments to diagnosing patients. As such, there are opportunities to address these and other deficiencies in nuclear imaging systems.

### SUMMARY

Systems and methods for training deep learning processes to reconstruct medical images based on histo-projection data (e.g., time-of-flight (TOF) histo-projection data), and for reconstructing medical images based on the trained deep learning processes, are disclosed.

In some embodiments, a computer-implemented method includes receiving image measurement data. The method also includes generating histo-projection data based on the image measurement data. Further, the method includes applying a trained machine learning process to the histo-projection data and, based on applying the trained machine learning process to the histo-projection data, generating a reconstructed image. The method also includes storing the reconstructed image in a data repository.

In some embodiments, a non-transitory computer readable medium stores instructions that, when executed by at least one processor, cause the at least one processor to perform operations. The operations include receiving image measurement data. The operations also include generating histo-projection data based on the image measurement data. Further, the operations include applying a trained machine learning process to the histo-projection data and, based on applying the trained machine learning process to the histo-projection data, generating a reconstructed image. The operations also include storing the reconstructed image in a data repository.

In some embodiments, a system includes a memory storing instructions and at least one processor communicatively coupled the memory. The at least one processor is configured to execute the instructions to receive image measurement data. The at least one processor is also configured to execute the instructions to generate histo-projection data based on the image measurement data. Further, the at least one processor is configured to execute the instructions to apply a trained machine learning process to the histo-projection data and, based on the application of the trained machine learning process to the histo-projection data, generate a reconstructed image. The at least one processor is also configured to execute the instructions to store the reconstructed image in a data repository.

In some embodiments, a computer-implemented method includes receiving histo-projection data. The method also includes inputting the histo-projection data to a machine learning process and, based on inputting the histo-projection data to the machine learning process, generating output data. Further, the method includes generating a loss value based on the output data and ground truth data. The method also includes determining the machine learning process is trained based on the loss value. The method further includes storing parameters associated with the machine learning process in a data repository.

In some embodiments, a non-transitory computer readable medium stores instructions that, when executed by at least one processor, cause the at least one processor to perform operations. The operations include receiving histo-projection data. The operations also include inputting the histo-projection data to a machine learning process and, based on inputting the histo-projection data to the machine learning process, generating output data. Further, the operations include generating a loss value based on the output data and ground truth data. The operations also include determining the machine learning process is trained based on the loss value. The operations further include storing parameters associated with the machine learning process in a data repository.

In some embodiments, an apparatus includes a memory storing instructions and at least one processor communicatively coupled the memory. The at least one processor is configured to execute the instructions to receive histo-projection data. The at least one processor is also configured to execute the instructions to input the histo-projection data to a machine learning process and, based on the input of the histo-projection data to the machine learning process, generate output data. Further, the at least one processor is configured to execute the instructions to generate a loss value based on the output data and ground truth data. The at least one processor is also configured to execute the instructions to determine the machine learning process is trained based on the loss value. The at least one processor is further configured to execute the instructions to store parameters associated with the machine learning process in a data repository.

### BRIEF DESCRIPTION OF THE DRAWINGS

The following will be apparent from elements of the figures, which are provided for illustrative purposes and are not necessarily drawn to scale.

FIG. 1 illustrates a nuclear image reconstruction system, in accordance with some embodiments.

FIG. 2 illustrates a block diagram of an example computing device that can perform one or more of the functions described herein, in accordance with some embodiments.

FIG. 3 illustrates a system that trains machine learning models, in accordance with some embodiments.

FIG. 4 illustrates the training of a machine learning model, in accordance with some embodiments.

FIGS. 5A and 5B illustrate exemplary image reconstruction processes using trained machine learning models, in accordance with some embodiments.

FIG. 6 is a flowchart of an example method to reconstruct image, in accordance with some embodiments.

FIG. 7 is a flowchart of an example method to train a machine learning process, in accordance with some embodiments.

FIG. 8 illustrates various reconstructed images.

FIG. 9 illustrates various reconstructed images.

### DETAILED DESCRIPTION

This description of the exemplary embodiments is intended to be read in connection with the accompanying drawings, which are to be considered part of the entire written description. Independent of the grammatical term usage, individuals with male, female, or other gender identities are included within the term.

The exemplary embodiments are described with respect to the claimed systems as well as with respect to the claimed methods. Furthermore, the exemplary embodiments are described with respect to methods and systems for image reconstruction, as well as with respect to methods and systems for training functions used for image reconstruction. Features, advantages, or alternative embodiments herein can be assigned to the other claimed objects and vice versa. For example, claims for the providing systems can be improved with features described or claimed in the context of the methods, and vice versa. In addition, the functional features of described or claimed methods are embodied by objective units of a providing system. Similarly, claims for methods and systems for training image reconstruction functions can be improved with features described or claimed in context of the methods and systems for image reconstruction, and vice versa.

Various embodiments of the present disclosure can employ machine learning methods or processes to provide clinical information from nuclear imaging systems. For example, the embodiments can employ machine learning methods or processes to reconstruct images based on captured measurement data, and provide the reconstructed images for clinical diagnosis. In some embodiments, machine learning methods or processes are trained to improve the reconstruction of images.

End-to-end deep learning image reconstruction has gained interest in recent years. For example, Fast PET techniques include the use of neural networks that operate on histo-images and attenuation maps to reconstruct images. These techniques, however, can have drawbacks. For example, Fast PET techniques can suffer from overly blurred reconstructed images. As the images can cause a patient to undergo additional imaging, or possibly lead to subpar diagnosis or even misdiagnosis. The embodiments described herein may address these and other image reconstruction issues and drawbacks.

In some embodiments, a machine learning model (e.g., machine learning algorithm), such as a neural network (e.g., convolutional neural network (CNN)), is trained based on histo-projection data. Histo-projection data characterizes histo-projections (e.g., projections extended in a corresponding TOF direction through an image space). The histo-projection data may be generated from image measurement data, such as sinogram data or list mode data generated by a Positron Emission Tomography (PET) scanning system. For instance, the histo-projection data may be generated based on histogramming list mode data (e.g., binned TOF events are histogrammed into “histo-projections”). The machine learning model may be iteratively trained with multiple epochs of histo-projection data. For instance, to train the machine learning model, the histo-projection data and corresponding ground truth data is input into the machine learning model. The ground truth data may characterize expected reconstructed images (e.g., corrected reconstructed images). Based on the inputted histo-projection data and ground truth data, the machine learning model generates output data characterizing a reconstructed image. Further, and based on execution of an optimization algorithm (e.g., gradient descent, Adaptive Moment Estimation, Broyden-Fletcher-Goldfarb-Shanno, stochastic optimization such as AdaGrad, or root mean square propagation, etc.), one or more weights associated with various layers of the machine learning model are adjusted to, for instance, minimize a difference between the reconstructed image and the corresponding ground truth data.

In some instances, the machine learning model is also trained with corresponding attenuation maps (e.g., μ-maps) generated from a co-modality scan, such as CT. In these examples, the optimization algorithm may adjust the weights of the machine learning model to generate the output data, where the machine learning model may apply the weights to input features from the inputted histo-projection data and their corresponding attenuation maps.

In some examples, rather than histo-projection data, the machine learning model is trained with back-projected images (e.g., back-projected histo-projections). For example, the back-projected images can be generated based on applying a back-projection process to the histo-projection data. The machine learning model may be iteratively trained with multiple epochs of back-projected images and corresponding ground truth data. For instance, to train the machine learning model, the back-projected images and corresponding ground truth data is input into the machine learning model.

The ground truth data may characterize expected reconstructed images (e.g., corrected reconstructed images). Based on the inputted back-projected images and ground truth data, the machine learning model generates output data characterizing a reconstructed image. Further, and based on execution of an optimization algorithm, one or more weights associated with various layers of the machine learning model are adjusted to, for instance, minimize a difference between the reconstructed image and the corresponding ground truth data. In some instances, the machine learning model is also trained with corresponding attenuation maps (e.g., μ-maps) generated from a co-modality scan, such as CT. In these examples, the optimization algorithm may adjust the weights of the machine learning model to generate the output data, where the machine learning model may apply the weights to input features from the inputted back-projected images and their corresponding attenuation maps.

In some examples, to determine whether training of the machine learning process is complete, a loss can be computed based on the output data and the ground truth data. The loss may be computed based on any suitable loss function (e.g., image reconstruction loss function), such as any of the mean square error (MSE), mean absolute error (MAE), binary cross-entropy (BCE), Sobel, Laplacian, and Focal binary loss functions. A determination may be made as to whether the machine learning model is trained based on the computed loss. For instance, if the computed loss at least meets (e.g., exceeds, is below) a corresponding loss threshold, then a determination is made that the machine learning model is trained. Otherwise, if the computed loss does not at least meet the loss threshold, a determination is made that the machine learning model is not trained. In this case, the machine learning model may be trained with further epochs of training data as described herein. The training of the machine learning model may continue until the loss at least meets the loss threshold.

In some examples, once the loss at least meets the loss threshold, the machine learning model may be validated using previously unused histo-projection data or back-projected images and, in some examples, corresponding attenuation maps. For example, additional histo-projection data or back-projected images may be inputted to the machine learning model to generate validation output images. A loss may be computed (e.g., using a loss function) based on the validation output images and corresponding ground truth images (e.g., ground truth reconstructed images). The machine learning model may be considered trained and validated when the computed loss at least meets a corresponding loss threshold. Otherwise, if the machine learning model does not validate, then the machine learning model may be further trained as described herein.

Once trained and, in some examples, validated, the trained machine learning model may be employed by image reconstruction systems to reconstruct images. For example, an image reconstruction system may receive PET measurement data (e.g., list mode data) from a PET/CT imaging system. The image reconstruction system may generate histo-projection data based on the PET measurement data. The image reconstruction system may then apply the trained machine learning model (e.g., the trained neural network) to the histo-projection data (and, in some examples, corresponding attenuation maps). Based on applying the trained machine learning process to the histo-projection data (and, in some examples, corresponding attenuation maps), the image reconstruction system may generate a reconstructed image, i.e., a final image volume.

In another example, the image reconstruction system may receive PET measurement data (e.g., list mode data) from the PET/CT imaging system, and may apply a back-projection process to the PET measurement data. Based on applying the back-projection process to the PET measurement data, the image reconstruction system may generate back-projected images. For instance, the image reconstruction system may apply a filtered back-projection (FBP) algorithm to the PET measurement data to generate the back-projected images. The image reconstruction system may then apply the trained machine learning model (e.g., the trained neural network) to the back-projected images (and, in some examples, corresponding attenuation maps).

Based on applying the trained machine learning process to the histo-projection data (and, in some examples, corresponding attenuation maps), the image reconstruction system can generate a reconstructed image, i.e., a final image volume.

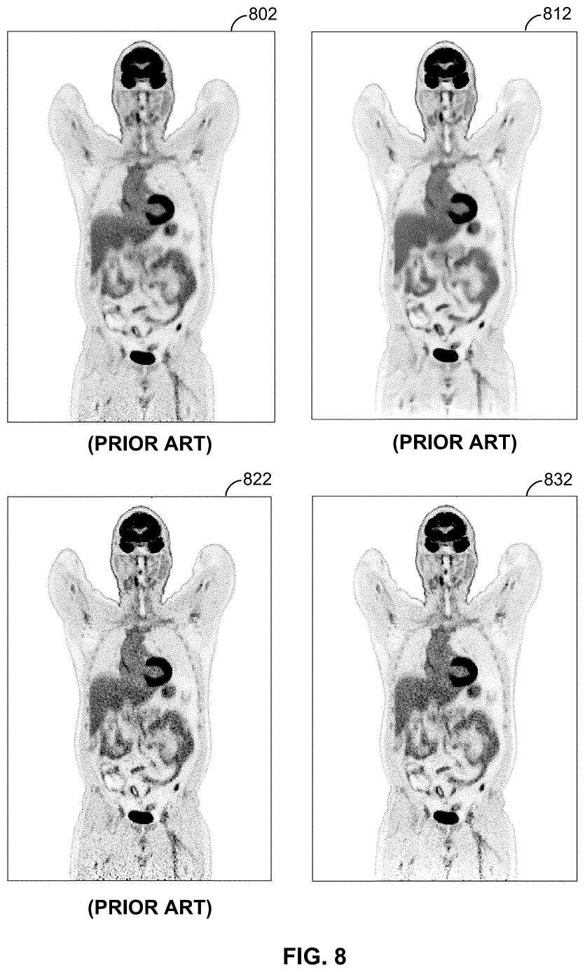

FIG. 8, for instance, illustrates various reconstructed images. The images of FIG. 8 illustrate images reconstructed from PET measurement data captured during whole-body scans. First reconstructed image 802 was generated based on a prior art Ordered Subset Expectation Maximization (OSEM) process. In addition, second reconstructed image 812 was generated based on a prior art deep learning histo-image process, and third reconstructed image 822 was generated based on a prior art Maximum Likelihood Expectation Maximization (MLEM) process. The fourth reconstructed image 832 was generated based on applying a trained machine learning process to histo-projection data characterizing back-projected images, as described herein. As illustrated, the fourth reconstructed image 832 is sharper than the first reconstructed image 802 and the second reconstructed image 812, and is closer to the targeted third reconstructed image 822 than are any of the first reconstructed image 802 and the second reconstructed image 812.

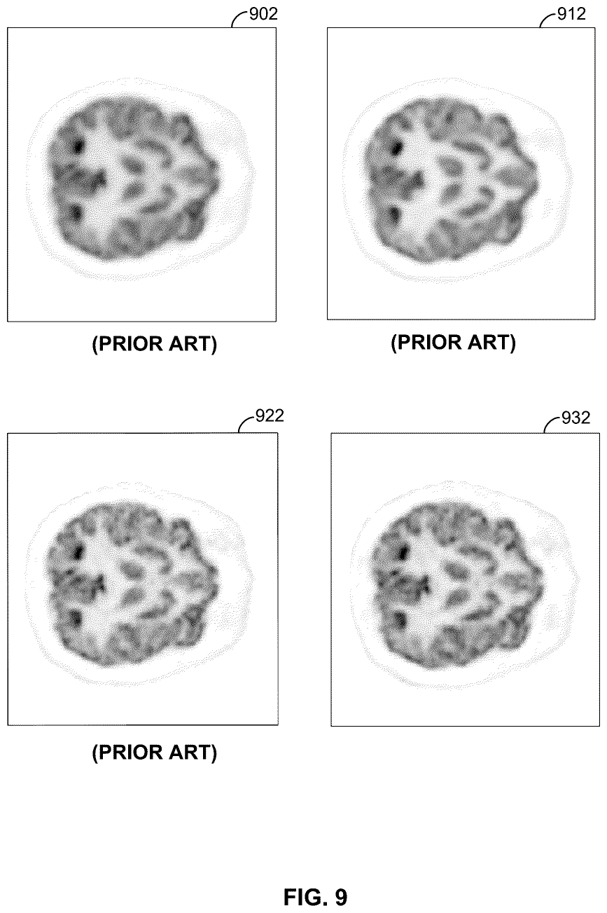

FIG. 9 also illustrates various reconstructed images. The images of FIG. 9 illustrate images reconstructed from PET measurement data captured during brain scans. First reconstructed image 902 was generated based on a prior art OSEM process. In addition, second reconstructed image 912 was generated based on a prior art deep learning histo-image process, and third reconstructed image 922 was generated based on a prior art MLEM process. The fourth reconstructed image 932 was generated based on applying a trained machine learning process to histo-projection data characterizing back-projected images, as described herein. As illustrated, the fourth reconstructed image 932 is sharper than the first reconstructed image 902 and the second reconstructed image 912, and is closer to the targeted third reconstructed image 922 than are any of the first reconstructed image 902 and the second reconstructed image 912.

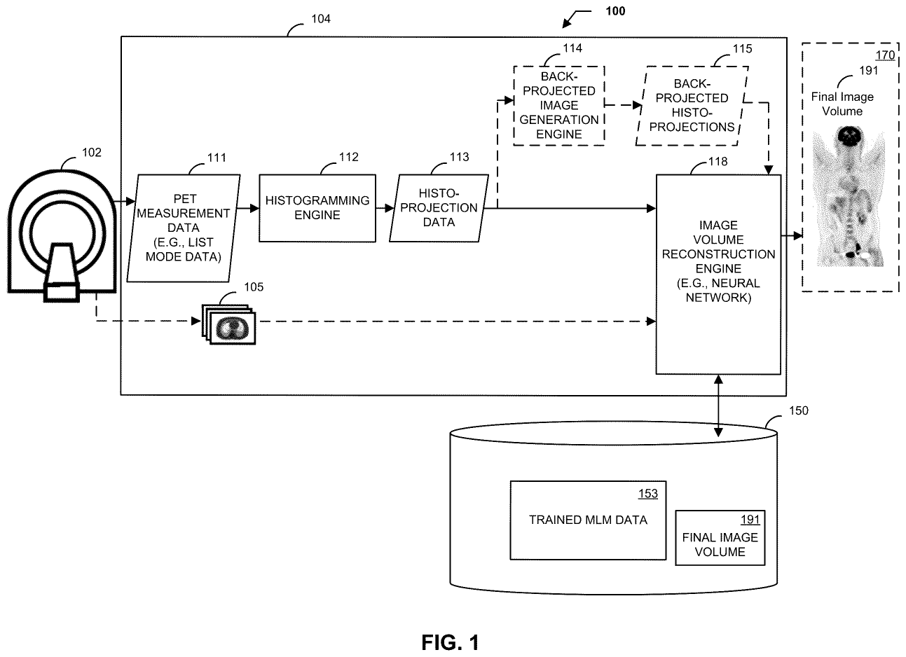

Referring now to the drawings, FIG. 1 illustrates a nuclear imaging system 100 that includes image scanning system 102 and image reconstruction system 104. Image scanning system 102 may be PET scanner that can capture PET images, a PET/MR scanner that can capture PET and MR images, a PET/CT scanner that can capture PET and CT images, or any other suitable image scanner. For example, as illustrated, image scanning system 102 can capture PET images (e.g., of a person), and can generate PET measurement data 111 (e.g., PET raw data, such as list mode data or sinogram data) based on the captured PET images. The PET measurement data 111 can represent anything imaged in the scanner's field-of-view (FOV) containing positron emitting isotopes. For example, the PET measurement data 111 can represent whole-body image scans, such as image scans from a patient's head to thigh. Further, image scanning system can transmit the PET measurement data 111 to image reconstruction system 104 (e.g., over one or more wired or wireless communication channels).

In some examples, image scanning system 102 may additionally generate attenuation maps 105 (e.g., μ-maps). For instance, the attenuation map 105 may be based on a separate scan of the patient without receiving radiotracer injections. In other examples, the image scanning system 102 may be a PET/CT scanner that, in addition to PET images, can capture CT scans of the patient. The image scanning system 102 may generate the attenuation maps 105 based on the captured CT images. As another example, the image scanning system 102 may be a PET/MR scanner that, in addition to PET images, can capture MR scans of the patient. The image scanning system 102 may generate the attenuation maps 105 based on the captured MR images. Further, the image scanning system 102 may transmit the attenuation maps 105 to the image reconstruction system 104.

In some examples, all or parts of image reconstruction system 104 are implemented in hardware, such as in one or more field-programmable gate arrays (FPGAs), one or more application-specific integrated circuits (ASICs), one or more state machines, one or more computing devices, digital circuitry, or any other suitable circuitry. In some examples, parts or all of image reconstruction system 104 can be implemented in software as executable instructions such that, when executed by one or more processors, cause the one or more processors to perform respective functions as described herein. The instructions can be stored in a non-transitory, computer-readable storage medium, and can be read and executed by the one or more processors.

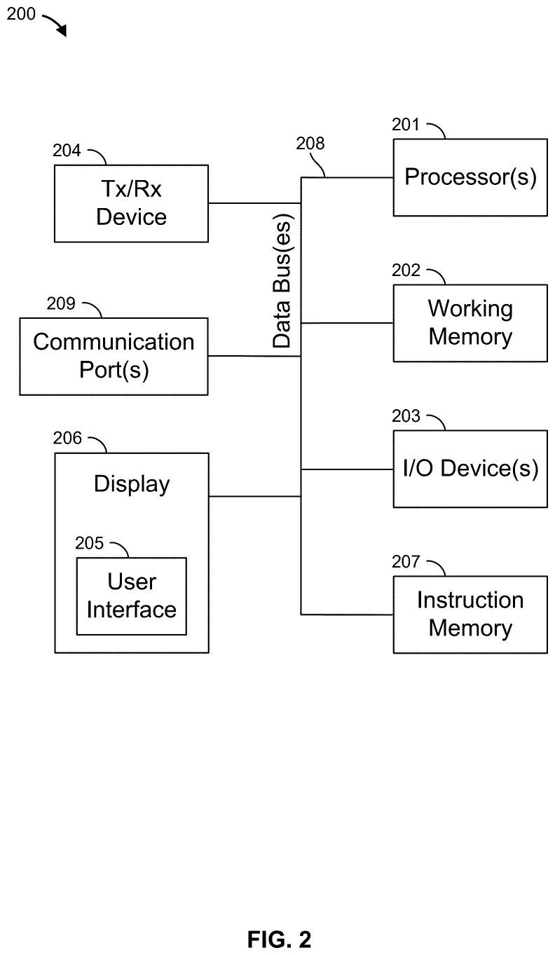

FIG. 2, for example, illustrates an image data processing device 200 that can be employed by the image reconstruction system 104. The image data processing device 200 can implement one or more of the functions of the image reconstruction system 104 described herein.

The image data processing device 200 can include one or more processors 201, working memory 202, one or more input/output devices 203, instruction memory 207, a transceiver 204, one or more communication ports 209, and a display 206, all operatively coupled to one or more data buses 208. Data buses 208 allow for communication among the various devices. Data buses 208 can include wired, or wireless, communication channels.

Processors 201 can include one or more distinct processors, each having one or more cores. Each of the distinct processors can have the same or different structure. Processors 201 can include one or more central processing units (CPUs), one or more graphics processing units (GPUs), application specific integrated circuits (ASICs), digital signal processors (DSPs), and the like.

Processors 201 can be configured to perform a certain function or operation by executing code, stored on instruction memory 207, embodying the function or operation. For example, processors 201 can be configured to perform one or more of any function, method, or operation disclosed herein.

Instruction memory 207 can store instructions that can be accessed (e.g., read) and executed by processors 201. For example, instruction memory 207 can be a non-transitory, computer-readable storage medium such as a read-only memory (ROM), an electrically erasable programmable read-only memory (EEPROM), flash memory, a removable disk, CD-ROM, any non-volatile memory, or any other suitable memory. For example, instruction memory 207 can store instructions that, when executed by one or more processors 201, cause one or more processors 201 to perform one or more of the functions of image reconstruction system 104, such as one or more of the machine learning processes and/or forward projection processes described herein.

Processors 201 can store data to, and read data from, working memory 202. For example, processors 201 can store a working set of instructions to working memory 202, such as instructions loaded from instruction memory 207. Processors 201 can also use working memory 202 to store dynamic data created during the operation of image data processing device 200. Working memory 202 can be a random access memory (RAM) such as a static random access memory (SRAM) or dynamic random access memory (DRAM), or any other suitable memory.

Input/output devices 203 can include any suitable device that allows for data input or output. For example, input/output devices 203 can include one or more of a keyboard, a touchpad, a mouse, a stylus, a touchscreen, a physical button, a speaker, a microphone, or any other suitable input or output device.

Communication port(s) 209 can include, for example, a serial port such as a universal asynchronous receiver/transmitter (UART) connection, a Universal Serial Bus (USB) connection, or any other suitable communication port or connection. In some examples, communication port(s) 209 allows for the programming of executable instructions in instruction memory 207. In some examples, communication port(s) 209 allow for the transfer (e.g., uploading or downloading) of data, such as PET measurement data 111 and/or attenuation maps 105.

Display 206 can display user interface 205. User interfaces 205 can enable user interaction with image data processing device 200. For example, user interface 205 can be a user interface for an application that allows for the viewing of final image volumes 191. In some examples, a user can interact with user interface 205 by engaging input/output devices 203. In some examples, display 206 can be a touchscreen, where user interface 205 is displayed on the touchscreen.

Transceiver 204 allows for communication with a network, such as a Wi-Fi network, an Ethernet network, a cellular network, or any other suitable communication network. For example, if operating in a cellular network, transceiver 204 is configured to allow communications with the cellular network. Processor(s) 201 is operable to receive data from, or send data to, a network via transceiver 204.

Referring back to FIG. 1, image reconstruction system 104 includes histogramming engine 112, image volume reconstruction engine 118, and, optionally, back-projected image generation engine 114. One or more of histogramming engine 112, back-projected image generation engine 114 and image volume reconstruction engine 118 may be implemented in hardware (e.g., digital logic), or by one or more processors, such as processor 201, executing instructions, or in any combination thereof.

As illustrated, histogramming engine 112 operates on PET measurement data 111 to generate histo-projection data 113. For instance, histogramming engine 112 may be a histogrammer that generates histo-projections based on list mode data.

Further, image volume reconstruction engine 118 receives the histo-projection data 113, and applies a trained machine learning process to the histo-projection data 113 to reconstruct a corresponding final image volume 191. For example, image volume reconstruction engine 118 may input the histo-projection data 113 to a trained neural network that generates, based on the inputted histo-projection data 113, the final image volume 191. Image volume reconstruction engine 118 may store the final image volume 191 in data repository 150. In some instances, image volume reconstruction engine 118 may display the final image volume 191 within a display 170

In some examples, image volume reconstruction engine 118 also receives from the image scanning system 102 an attenuation map 105 corresponding to the PET measurement data 111. The image volume reconstruction engine 118 may apply the trained machine learning process to the histo-projection data 113 and the attenuation map 105 to generate the final image volume 191. For instance, image volume reconstruction engine 118 may parse the attenuation map 105 to extract attenuation correction values, and may adjust corresponding values within the histo-projection data 113 to generate the final image volume 191. In this example, the final image volume 191 is an attenuation corrected reconstructed PET image.

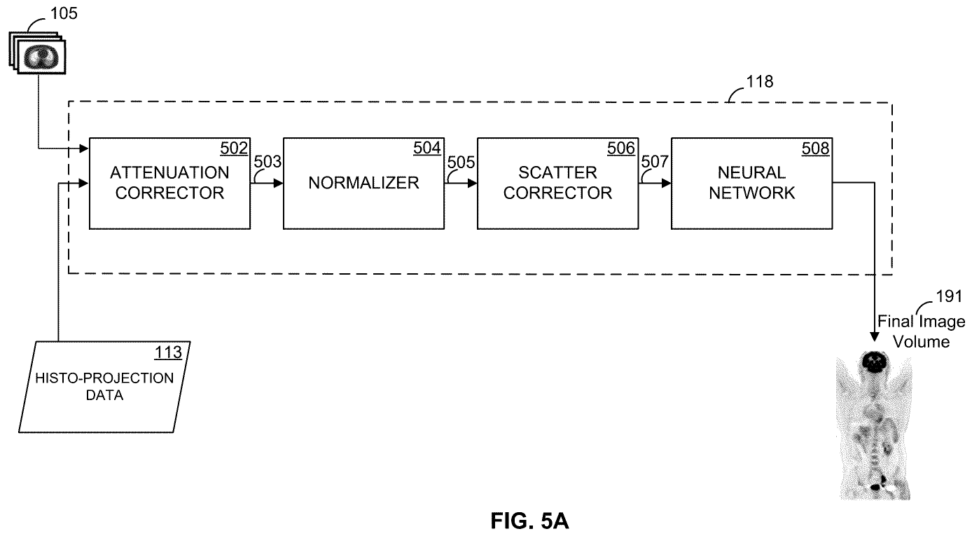

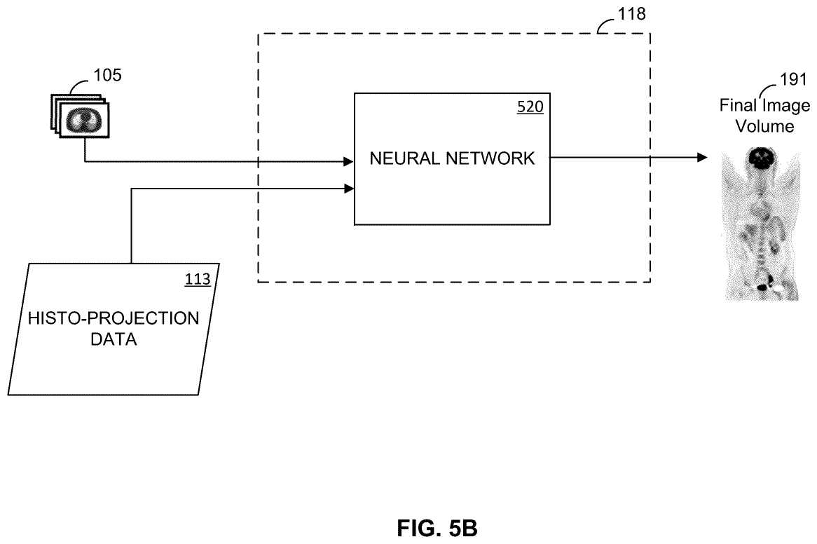

For instance, FIG. 5A illustrates an example of the image volume reconstruction engine 118 that includes an attenuation corrector 502, a normalizer 504, a scatter corrector 506, and a trained neural network 508. Attenuation correction 502 executes an attenuation correction process to correct the histo-projection data 113 based on the attenuation map 105, thereby generating attenuation corrected histo-projection data 503. Further, the normalizer 504 performs operations to normalize the attenuation corrected histo-projection data 503, and provides normalized histo-projection data 505 to the scatter corrector 506. The scatter corrector 506 performs operations to the normalized histo-projection data 505 to correct for scatter and random coincidences, thereby generating input histo-projection image data 507. The image volume reconstruction engine 118 inputs the input histo-projection image data 507 to the trained neural network 508 and, in response, generates the final image volume 191.

FIG. 5B illustrates another example of the image volume reconstruction engine 118. In this example, the image volume reconstruction engine 118 includes a trained neural network 520 that is configured to receive the histo-projection data 113 and the corresponding attenuation map 105, and inputs the histo-projection data 113 and the corresponding attenuation map 105 to the trained neural network 520. In response, the trained neural network 520 generates the final image volume 191. In this example, the trained neural network is trained (e.g., using corrected reconstructed images as ground truth data) to correct for attenuation and/or scatter and, as such, the final image volume 191 may be attenuation and/or scatter corrected. For instance, in some examples, the trained neural network is trained to correct histo-projection data 113 for scatter, where the inputted histo-projection data 113 is already corrected for attenuation.

Referring back to FIG. 1, as described herein, applying the trained machine learning process to the histo-projection data 113 and, in some examples, the corresponding attenuation maps 105, can include generating input features based on the histo-projection data 113 and/or the attenuation maps 105, and inputting the generated features to a trained machine learning model, such as a trained neural network. Based on the inputted features, the trained machine learning model generates output data characterizing the final image volume 191.

In some examples, image volume reconstruction engine 118 reconstructs final image volumes 191 based on back-projected images. For instance, back-projected image generation engine 114 can generate back-projected histo-projections 115 based on the histo-projection data 113 using any suitable back-projection method known in the art. The back-projection process can be a transpose operation of a forward projection process. For instance, in the case of time-of-flight (TOF), a back-projection can include a convolution with measured width TOF kernel. The TOF backprojection can include a TOF kernel with any suitable width, including a spatially varying TOF kernel.

Further, image volume reconstruction engine 118 receives each back-projected histo-projection 115 and applies a trained machine learning process to the back-projected histo-projection 115 to reconstruct a corresponding final image volume 191. For example, image volume reconstruction engine 118 may input the back-projected histo-projection 115 to a trained neural network that generates, based on the inputted back-projected histo-projection 115, the final image volume 191. Image volume reconstruction engine 118 may store the final image volume 191 in data repository 150. In some instances, image volume reconstruction engine 118 may display the final image volume 191 within a display 170.

In some examples, image volume reconstruction engine 118 also receives the attenuation map 105 from the image scanning system 102, and applies the trained machine learning process to the back-projected histo-projection 115 and the attenuation map 105 to generate the final image volume 191. For instance, image volume reconstruction engine 118 may parse the attenuation map 105 to extract attenuation correction values, and may adjust corresponding values within back-projected histo-projection 115 to generate final image volume 191. In this example, the final image volume 191 is an attenuation corrected reconstructed PET image.

As described herein, applying the trained machine learning process to the back-projected histo-projections 115 and, in some examples, the corresponding attenuation maps 105, can include generating input features based on the back-projected histo-projections 115 and/or the attenuation maps 105, and inputting the generated features to a trained machine learning model, such as a trained neural network. Based on the inputted features, the trained machine learning model generates output data characterizing the final image volume 191.

To establish any of the trained machine learning models described herein, the image reconstruction system 104 may obtain, from data repository 150, trained machine learning model (MLM) data 153, which includes parameters (e.g., coefficients, weights, etc.) characterizing the trained machine learning model. For example, the image reconstruction system 104 may configure an executable machine learning model (e.g., executable instructions characterizing the trained machine learning model) based on (e.g., with) the parameters of the trained MLM data 153 to establish the trained machine learning model (e.g., the trained neural network). As described further herein, the trained MLM data 153 is generated based on training and, in some examples, validating, a machine learning model using back-projected images.

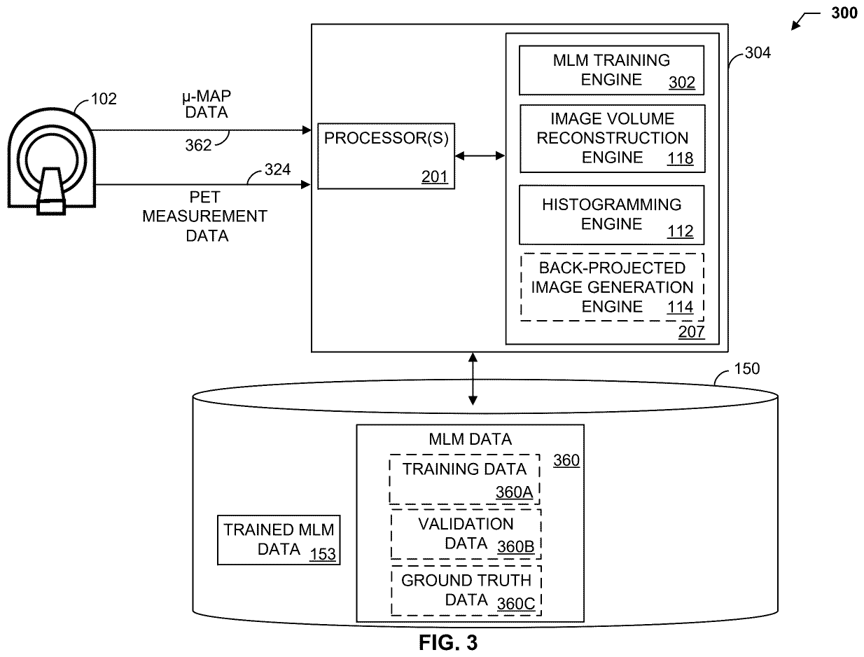

FIG. 3 illustrates a training system 300 that can train a machine learning model, such as a neural network, based on back-projected images to generate a reconstructed image, such as the final image volume 191 of FIG. 1. The training system 300 includes an image data processing device 304, the image scanning system 102, and the data repository 150.

In this example, the instruction memory 207 includes executable instructions for an MLM training engine 302, the image volume reconstruction engine 118, and the back-projected image generation engine 114. Further, one or more processors 201 are communicatively coupled to the instruction memory 207, and are configured to execute any one or more of the MLM training engine 302, the image volume reconstruction engine 118, and the back-projected image generation engine 114.

As illustrated, the image data processing device 304 is communicatively coupled to data repository 150 and to image scanning system 102. Data repository 150 may store MLM data 360, which may include training data 360A, validation data 360B, and/or ground truth data 360C, for instance. Training data 360A may include epochs of histo-projection data (e.g., histo-projection data 113) and/or back-projected images (e.g., back-projected histo-projections 115) to be used for training a machine learning model. For example, the histo-projection data and/or back-projected images of the training data 360A may be generated based on PET measurement data 324 received from image scanning system 102, as described herein. In some instances, training data 360A further includes corresponding attenuation maps. The attenuation maps may be based on μ-map data 362 received from image scanning system 102. Further, validation data 360B may include epochs of histo-projection data and/or back-projected images to be used for validating (e.g., testing) an initially trained machine learning model. In some instances, validation data 360B also includes corresponding attenuation maps. In some examples, the training data 360A and validation data 360B include distinct epochs of histo-projection data and/or back-projected images.

Executed MLM training engine 302 may obtain training data 360A from data repository 150, and may generate features based on the training data 360A (e.g., features of histo-projection data and/or back-projected images and, in some examples, features of corresponding attenuation maps). Further, executed MLM training engine 302 may input the features to an untrained machine learning model that, in response, generates output image data. The output image data may characterize a reconstructed image. Further, executed MLM training engine 302 may compute a loss value based on the output image data and the ground truth data 360C characterizing expected reconstructed PET images. For instance, executed based MLM training engine 302 may compute the loss value based on a loss function, such as any of the MSE, MAE, BCE, Sobel, Laplacian, and Focal binary loss functions, or any other suitable loss function.

Based on the computed loss value, executed MLM training engine 302 may determine whether the machine learning model is trained. For instance, executed MLM training engine 302 may compare the loss value to a corresponding loss threshold value to determine if the loss value at least meets the corresponding loss threshold value. If the loss value at least meets the corresponding loss threshold value, the executed MLM training engine 302 may determine the machine learning model is trained, and may store parameters associated with the now trained machine learning model as trained MLM data 153 within data repository 150. Otherwise, if the loss value does not at least meet the corresponding loss threshold value, the executed MLM training engine 302 may perform operations to continue training the machine learning model.

In some instances, once the machine learning model is trained, executed MLM training engine 302 may perform operations to validate the initially trained machine learning model. For example, executed MLM training engine 302 may obtain validation data 360B from the data repository 150, and may generate features based on the validation data 360B. Further, executed MLM training engine 302 may input the generated validation features to the initially trained machine learning model and, in response to the inputted validation features, generates additional output image data. Further, executed MLM training engine 302 may compute an additional loss value based on the additional output image data and the ground truth data 360C corresponding to the validation data 360B.

Based on the computed additional loss value, executed MLM training engine 302 may determine whether the machine learning model is validated. For instance, executed MLM training engine 302 may compare the additional loss value to a corresponding loss threshold value to determine if the additional loss value at least meets the corresponding loss threshold value. If the additional loss value at least meets the corresponding loss threshold value, the executed MLM training engine 302 may determine the machine learning model is trained and validated, and may store parameters associated with the now trained and validated machine learning model as trained MLM data 153 within data repository 150. Otherwise, if the additional loss value does not at least meet the corresponding loss threshold value, the executed MLM training engine 302 may perform operations to continue training, and validating, the machine learning model.

Once trained, the machine learning process can generate reconstructed images, such as the final image volume 191, based on histo-projection data and/or attenuation maps, or based on back-projected images and/or attenuation maps, as described herein.

For instance, to reconstruct images based on histo-projection data and/or attenuation maps, the executed histogramming engine 112 may apply a histogramming process to PET measurement data 324 received from the image scanning system 102, and may generate histo-projection data (e.g., histo-projection data 113). Further, the executed image volume reconstruction engine 118 may input the histo-projection data and, in some examples, the μ-map data 362, to the trained machine learning model and, in response, generate a final image volume, such as the final image volume 191 of FIG. 1. In some examples, the executed image volume reconstruction engine 118 may input the histo-projection data and corresponding μ-map data 362 (received from the image scanning system 102) to the trained machine learning model and, in response, generate the final image volume.

In other examples, to reconstruct images based on back-projected images and/or attenuation maps, the executed back-projected image generation engine 114 may apply a back-projection process to histo-projection data 113 received from the histogramming engine 112. Further, the executed image volume reconstruction engine 118 may input the back-projected images and, in some examples, the μ-map data 362, to the trained machine learning model and, in response, generate a final image volume, such as the final image volume 191 of FIG. 1. In some examples, the executed image volume reconstruction engine 118 may input the back-projected images and corresponding μ-map data 362 (received from the image scanning system 102) to the trained machine learning model and, in response, generate the final image volume.

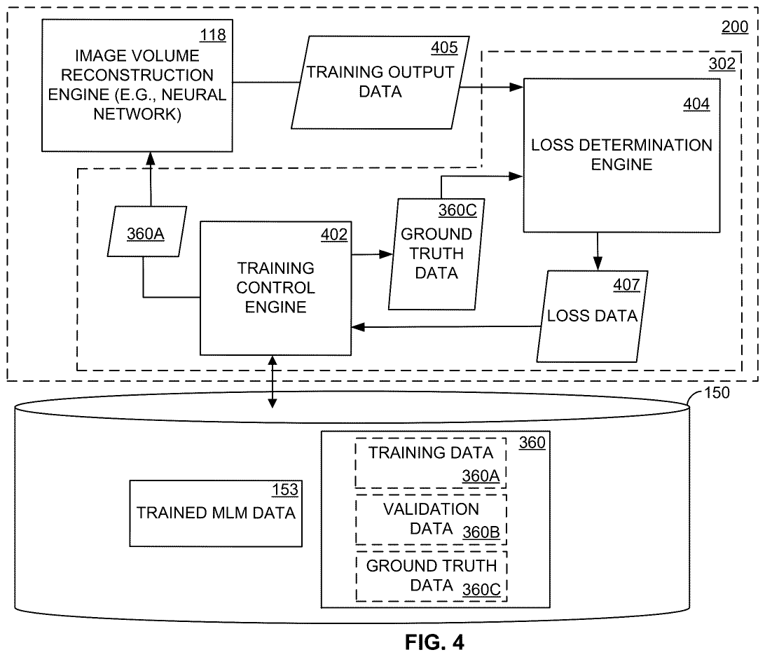

FIG. 4 illustrates an example of the MLM training engine 302 that can train a machine learning model of the image volume reconstruction engine 118. The MLM training engine 302 can include a training control engine 402 and a loss determination engine 404. In this example, the training control engine 402 obtains (e.g., receives) training data 360A and corresponding ground truth data 360C from data repository 150, and provides (e.g., transmits) the training data 360A and the ground truth data 360C to the image volume reconstruction engine 118. As described herein, the training data 360A may include histo-projection data or back-projected images and, in some examples, corresponding attenuation maps, while the ground truth data 360C characterizes expected reconstructed images.

In addition, the image volume reconstruction engine 118 includes a machine learning model (e.g., a deep learning neural network such as a CNN) that is to be trained. The image volume reconstruction engine 118 receives the training data 360A, and generates input features based on the training data 360A. Further, the image volume reconstruction engine 118 inputs the generated input features to the untrained machine learning model and, in response to the inputted features, generates output data characterizing a reconstructed image. Based on execution of an optimization algorithm that operates on the output data and the corresponding ground truth data 360C, the image volume reconstruction engine 118 adjusts one or more weights of the untrained machine learning model. For instance, the optimization algorithm may attempt to reduce or minimize a difference in values between the output data and the corresponding ground truth data 360C. The training control engine 402 may train the machine learning model with a number of epochs of training data 360A.

When the training control engine 402 has completed training the machine learning model with the epochs of training data 360A, the training control engine 402 may provide additional training data 360A to the image volume reconstruction engine 118 and, in response, the image volume reconstruction engine 118 generates training output data 405 characterizing a reconstructed image. The loss determination engine 404 may receive the training output data 405 from the image volume reconstruction engine 118, as well as corresponding ground truth data 360C from the training control engine 402. The loss determination engine 404 determines loss data 407 characterizing a training loss value based on the training output data 405 and the ground truth data 360C. For instance, the loss determination engine 404 may compute the training loss value based on any suitable loss function (e.g., image reconstruction loss function), such as a MSE, MAE, BCE, Sobel, Laplacian, and Focal binary loss functions.

The training control engine 402 may receive the loss data 407 from the loss determination engine 404, and determines whether the machine learning model is trained based on the loss data 407. For example, the training control engine 402 may determine whether the loss value characterized by the loss data 407 at least meets a corresponding loss threshold. If the loss value at least meets the loss threshold, the training control engine 402 may determine that the machine learning model is trained. Otherwise, if the loss value does not meet the loss threshold, the training control engine 402 may continue to train the machine learning model with additional epochs of training data 360A.

In some examples, when the training control engine 402 determines that the machine learning model is trained, the training control engine 402 validates the machine learning model. For example, the training control engine 402 may obtain from the data repository 150 validation data 360B, and transmits the validation data 360B to the image volume reconstruction engine 118. The image volume reconstruction engine 118 receives the validation data 360B, and generates input features based on the validation data 360B. Further, the image volume reconstruction engine 118 inputs the generated input features to the machine learning model and, in response to the inputted features, generates training output data 405 characterizing a reconstructed image. Further, the loss determination engine 404 receives the training output data 405 from the image volume reconstruction engine 118, as well as corresponding ground truth data 360C from the training control engine 402. The loss determination engine 404 determines loss data 407 characterizing a validation loss value based on the training output data 405 and the ground truth data 360C.

The training control engine 402 may receive the loss data 407 from the loss determination engine 404, and determines whether the machine learning model is validated based on the loss data 407. For example, the training control engine 402 may determine whether the loss value characterized by the loss data 407 at least meets a corresponding loss threshold. If the loss value at least meets the loss threshold, the training control engine 402 may determine that the machine learning model is validated. Otherwise, if the loss value does not meet the loss threshold, the training control engine 402 may continue to train the machine learning model with additional epochs of training data 360A, and validate the machine learning model based on validation data 360B as described herein.

When the training control engine 402 determines the machine learning model is trained and, in some examples, validated, the training control engine 402 obtains trained machine learning model data 153 from the image volume reconstruction engine 118, where the trained machine learning model data 153 includes parameters (e.g., weights, coefficients, hyperparameters, etc.) associated with the trained machine learning model. The training control engine 402 then stores the trained machine learning model data 153 within data repository 150.

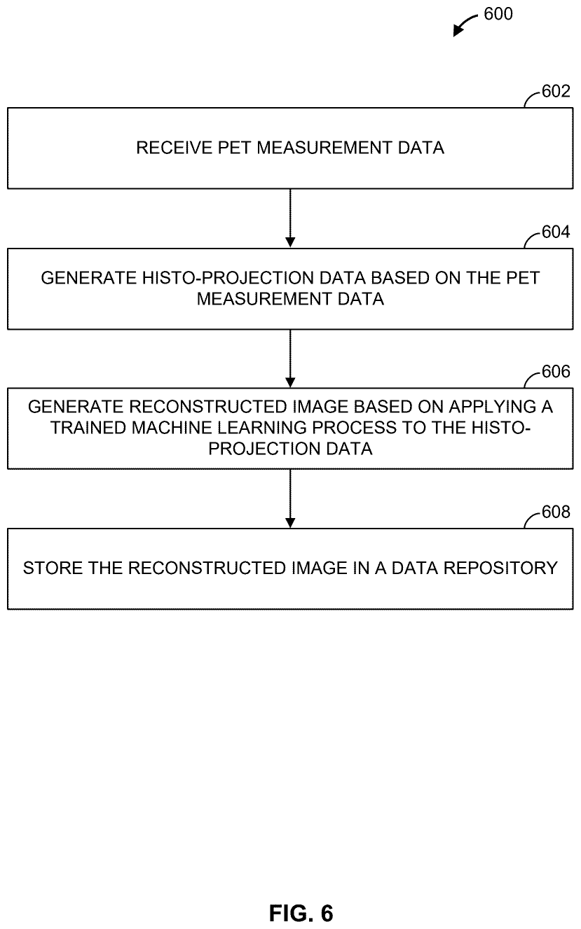

FIG. 6 is a flowchart of an example method 600 to reconstruct an image. The method can be performed by one or more image data processing devices, such as the image reconstruction system 104 or the image data processing device 200 (e.g., executing corresponding instructions).

Beginning at block 602, PET measurement data is received. The PET measurement data may be, for instance, PET measurement data 111 received from an image scanning system 102. The PET measurement data may include, for instance, list mode data or sinogram data. At block 604, histo-projection data is generated based on the PET measurement data. For instance, the image reconstruction system 104 can generate the histo-projection data based on applying a histogramming process the PET measurement data.

Proceeding to block 606, a trained machine learning process is applied to the histo-projection data. Based on the application of the trained machine learning process to the histo-projection data, a reconstructed image is generated. For example, and as described herein, the image reconstruction system 104 can establish (e.g., configure), based on the trained MLM data 153 stored in the data repository 150, a trained neural network that is configured to generate output data characterizing a reconstructed image. The image reconstruction system 104 can input the histo-projection data to the trained neural network and, based on inputting the histo-projection data to the trained neural network, can generate output data characterizing the reconstructed image, such as the final image volume 191.

At block 608, the reconstructed image is stored in a data repository. For instance, the image reconstruction system 104 may store the final image volume 191 in the data repository 150. In some examples, the image reconstruction system 104 provides the reconstructed image for display. In some examples, the image reconstruction system 104 transmits the reconstructed image to a receiving device (e.g., server), causing the receiving device to store the reconstructed image in a memory device (e.g., a cloud-accessible memory device).

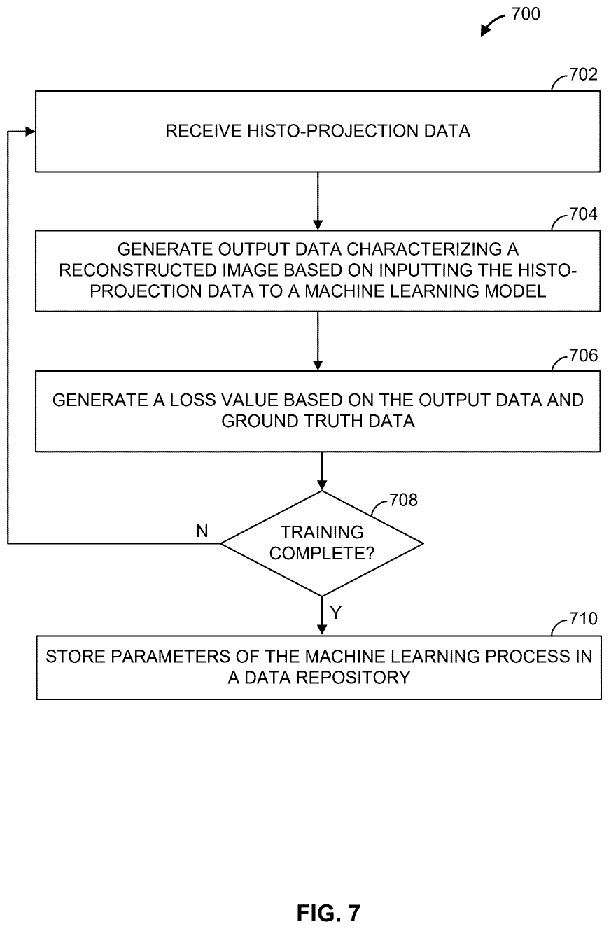

FIG. 7 is a flowchart of an example method 700 to train a machine learning process based on histo-projection data characterizing back-projected histo-projections. The method can be performed by one or more image data processing devices, such as the image reconstruction system 104 or the image data processing device 200 (e.g., executing corresponding instructions).

Beginning at block 702, histo-projection data is received. For example, the image data processing device 200 may obtain training data 360A from the data repository 150, where the training data 360A includes histo-projections. At block 704, the histo-projection data is inputted to a machine learning model and, based on inputting the histo-projection data to the machine learning model, output data is generated. The output data characterizes a reconstructed image during training. For example, as described herein, the image data processing device 200 may generate training output data 405 based on inputting portions of training data 360A to a neural network being trained.

In some instances, ground truth data, such as ground truth data 360C, is input to the machine learning model. The ground truth data characterizes expected reconstructed images based on corresponding portions of the training data. A corresponding optimization function can adjust weights of the in-training machine learning model based on the output data and the ground truth data, as described herein.

Proceeding to block 706, a loss value is generated based on the output data and ground truth data. For instance, and as described herein, the image data processing device 200 may obtain ground truth data 360C from the data repository 150. For instance, the image data processing device 200 may compute the loss value based on any suitable loss function (e.g., image reconstruction loss function), such as a MSE, MAE, BCE, Sobel, Laplacian, and Focal binary loss functions.

Proceeding to block 708, a determination is made as to whether training is complete. For instance, the image data processing device 200 may compare the loss value to a corresponding loss threshold value, and determine whether training is complete based on the determination. For example, if the loss value does not meet or exceed the corresponding loss threshold value, the machine learning model is not yet trained, and the method proceeds back to block 702 to continue its training. If, however, the loss value does meet or exceed the corresponding loss threshold value, the machine learning model is trained, and the method proceeds to block 710.

At block 710, parameters associated with the now trained machine learning model are stored in a data repository. For instance, the image data processing device 200 may store the parameters as trained MLM data 153 within data repository 150. As described herein, the image data processing device 200 may establish the trained machine learning model based on the stored parameters. Once established, the image data processing device 200 may generate reconstruct images, such as the final image volume 191, based on inputting back-projected images to the trained machine learning model.

The following is a list of non-limiting illustrative embodiments disclosed herein:

Illustrative Embodiment 1: A computer-implemented method comprising:
receiving image measurement data;
generating histo-projection data based on the image measurement data;
applying a trained machine learning process to the histo-projection data and, based on applying the trained machine learning process to the histo-projection data, generating a reconstructed image; and
storing the reconstructed image in a data repository.

Illustrative Embodiment 2: The computer-implemented method of illustrative embodiment 1, wherein applying the trained machine learning process to the histo-projection data comprises generating features based on the histo-projection data, and inputting the features to a trained machine learning model.

Illustrative Embodiment 3: The computer-implemented method of any of illustrative embodiments 1-2, wherein the trained machine learning model is a trained neural network.

Illustrative Embodiment 4: The computer-implemented method of any of illustrative embodiments 1-3, further comprising:
receiving an attenuation map; and
applying the trained machine learning process to the attenuation map and, based on applying the trained machine learning process to the attenuation map, generating the reconstructed image.

Illustrative Embodiment 5: The computer-implemented method of any of illustrative embodiments 1-4, further comprising applying an attenuation correction process to the histo-projection data to correct for attenuation, and applying the trained machine learning process to the attenuation corrected histo-projection data.

Illustrative Embodiment 6: The computer-implemented method of illustrative embodiment 5, further comprising receiving an attenuation map, and applying the attenuation correction process to the histo-projection data based on the attenuation map.

Illustrative Embodiment 7: The computer-implemented method of any of illustrative embodiments 1-6, further comprising generating the histo-projection data based on histogramming the image measurement data.

Illustrative Embodiment 8: The computer-implemented method of any of illustrative embodiments 1-7, wherein the trained machine learning process is trained with histo-projection data and corresponding ground truth data characterizing reconstructed images.

Illustrative Embodiment 9: The computer-implemented method of any illustrative embodiments 1-8, wherein the image measurement data comprises list mode data.

Illustrative Embodiment 10: The computer-implemented method of any of illustrative embodiments 1-9, wherein the image measurement data is positron emission tomography (PET) image data, and is received from at least one of a: PET scanner, PET/MR scanner, and PET/CT scanner.

Illustrative Embodiment 11: A computer-implemented method comprising:
receiving histo-projection data;
inputting the histo-projection data to a machine learning process and, based on inputting the histo-projection data to the machine learning process, generating output data;
generating a loss value based on the output data and ground truth data;
determining the machine learning process is trained based on the loss value; and
storing parameters associated with the machine learning process in a data repository.

Illustrative Embodiment 12: The computer-implemented method of illustrative embodiment 11, further comprising:
comparing the loss value to a loss threshold value; and
determining the machine learning process is trained based on the comparison.

Illustrative Embodiment 13: The computer-implemented method of any of illustrative embodiments 11-12, further comprising:
receiving image measurement data from an image scanning system; and
generating the histo-projection data based on the image measurement data.

Illustrative Embodiment 14: The computer-implemented method of any of illustrative embodiments 11-13, further comprising:
receiving attenuation maps corresponding to the back-projected images; and
inputting the attenuation maps to the machine learning process and, based on inputting the attenuation maps to the machine learning process, generating the output data.

Illustrative Embodiment 15: The computer-implemented method of any of illustrative embodiments 11-14, further comprising:
based on determining the machine learning process is trained:
   receiving additional histo-projection data;
   inputting the additional histo-projection data to the machine learning process and, based on inputting the additional histo-projection data to the machine learning process, generating additional output data;
   generating an additional loss value based on the additional output data and additional ground truth data; and
   determining the machine learning process is validated based on the additional loss value.

Illustrative Embodiment 16: An apparatus comprising:
a memory storing instructions; and
at least one processor communicatively coupled the memory, wherein the at least one processor is configured to execute the instructions to:
   receive image measurement data;
   generate histo-projection data based on the image measurement data;
   apply a trained machine learning process to the histo-projection data and, based on the application of the trained machine learning process to the histo-projection data, generate a reconstructed image; and
   store the reconstructed image in a data repository.

Illustrative Embodiment 17: The apparatus of illustrative embodiment 16,wherein, to apply the trained machine learning process to the histo-projection data, the at least one processor is configured to execute the instructions to:
generate features based on the histo-projection data; and
input the features to a trained machine learning model.

Illustrative Embodiment 18: The apparatus of any of illustrative embodiments 16-17, wherein the trained machine learning model is a trained neural network.

Illustrative Embodiment 19: The apparatus of any of illustrative embodiments 16-18, wherein the at least one processor is configured to execute the instructions to:
receive an attenuation map; and
apply the trained machine learning process to the attenuation map and, based on the application of the trained machine learning process to the attenuation map, generate the reconstructed image.

Illustrative Embodiment 20: The apparatus of any of illustrative embodiments 16-19, wherein the at least one processor is configured to execute the instructions to:
apply an attenuation correction process to the histo-projection data to correct for attenuation; and
apply the trained machine learning process to the attenuation corrected histo-projection data.

Illustrative Embodiment 21: The apparatus of illustrative embodiment 20, wherein the at least one processor is configured to execute the instructions to receive an attenuation map, and apply the attenuation correction process to the histo-projection data based on the attenuation map.

Illustrative Embodiment 22: The apparatus of any of illustrative embodiments 16-21, wherein the at least one processor is configured to execute the instructions to generate the histo-projection data based on histogramming the image measurement data.

Illustrative Embodiment 23: The apparatus of any of illustrative embodiments 16-22, wherein the trained machine learning process is trained with histo-projection data and corresponding ground truth data characterizing reconstructed images.

Illustrative Embodiment 24: The apparatus of any of illustrative embodiments 16-23, wherein the image measurement data comprises list mode data.

Illustrative Embodiment 25: The apparatus of any of illustrative embodiments 16-24, wherein the image measurement data is positron emission tomography (PET) image data, and is received from at least one of a: PET scanner, PET/MR scanner, and PET/CT scanner.

Illustrative Embodiment 26: An apparatus comprising:
a memory storing instructions; and
at least one processor communicatively coupled the memory, wherein the at least one processor is configured to execute the instructions to:
   receive histo-projection data;
   input the histo-projection data to a machine learning process and, based on inputting the histo-projection data to the machine learning process, generating output data;
   generate a loss value based on the output data and ground truth data;
   determine the machine learning process is trained based on the loss value; and
   store parameters associated with the machine learning process in a data repository.

Illustrative Embodiment 27: The apparatus of illustrative embodiment 26, wherein the at least one processor is configured to execute the instructions to:
compare the loss value to a loss threshold value; and
determine the machine learning process is trained based on the comparison.

Illustrative Embodiment 28: The apparatus of any of illustrative embodiments 26-27, wherein the at least one processor is configured to execute the instructions to:
receive image measurement data from an image scanning system; and
generate the histo-projection data based on the image measurement data.

Illustrative Embodiment 29: The apparatus of any of illustrative embodiments 26-28, wherein the at least one processor is configured to execute the instructions to:
receive attenuation maps corresponding to the back-projected images; and
input the attenuation maps to the machine learning process and, based on the input of the attenuation maps to the machine learning process, generate the output data.

Illustrative Embodiment 30: The apparatus of any of illustrative embodiments 26-29, wherein the at least one processor is configured to execute the instructions to
based on the determination that the machine learning process is trained:
   receive additional histo-projection data;
   input the additional histo-projection data to the machine learning process and, based on inputting the additional histo-projection data to the machine learning process, generating additional output data;
   generate an additional loss value based on the additional output data and additional ground truth data; and
determine the machine learning process is validated based on the additional loss value.

The apparatuses and processes are not limited to the specific embodiments described herein. In addition, components of each apparatus and each process can be practiced independent and separate from other components and processes described herein.

The previous description of embodiments is provided to enable any person skilled in the art to practice the disclosure. The various modifications to these embodiments will be readily apparent to those skilled in the art, and the generic principles defined herein can be applied to other embodiments without the use of inventive faculty. The present disclosure is not intended to be limited to the embodiments shown herein, but is to be accorded the widest scope consistent with the principles and novel features disclosed herein.

What is claimed is:

1. A computer-implemented method comprising:
receiving image measurement data;
generating histo-projection data based on the image measurement data;
applying a trained machine learning process to the histo-projection data and, based on applying the trained machine learning process to the histo-projection data, generating a reconstructed image; and
storing the reconstructed image in a data repository.

2. The computer-implemented method of claim 1, wherein applying the trained machine learning process to the histo-projection data comprises generating features based on the histo-projection data, and inputting the features to a trained machine learning model.

3. The computer-implemented method of claim 1, wherein the trained machine learning model is a trained neural network.

4. The computer-implemented method of claim 1, further comprising:
receiving an attenuation map; and
applying the trained machine learning process to the attenuation map and, based on applying the trained machine learning process to the attenuation map, generating the reconstructed image.

5. The computer-implemented method of claim 1, further comprising applying an attenuation correction process to the histo-projection data to correct for attenuation, and applying the trained machine learning process to the attenuation corrected histo-projection data.

6. The computer-implemented method of claim 5, further comprising receiving an attenuation map, and applying the attenuation correction process to the histo-projection data based on the attenuation map.

7. The computer-implemented method of claim 1, further comprising generating the histo-projection data based on histogramming the image measurement data.

8. The computer-implemented method of claim 1, wherein the trained machine learning process is trained with histo-projection data and corresponding ground truth data characterizing reconstructed images.

9. The computer-implemented method of claim 1, wherein the image measurement data comprises list mode data.

10. The computer-implemented method of claim 1, wherein the image measurement data is positron emission tomography (PET) image data, and is received from at least one of a: PET scanner, PET/MR scanner, and PET/CT scanner.

11. A computer-implemented method comprising:
receiving histo-projection data;
inputting the histo-projection data to a machine learning process and, based on inputting the histo-projection data to the machine learning process, generating output data;
generating a loss value based on the output data and ground truth data;
determining the machine learning process is trained based on the loss value; and
storing parameters associated with the machine learning process in a data repository.

12. The computer-implemented method of claim 11, further comprising:
comparing the loss value to a loss threshold value; and
determining the machine learning process is trained based on the comparison.

13. The computer-implemented method of claim 11, further comprising:
receiving image measurement data from an image scanning system; and
generating the histo-projection data based on the image measurement data.

14. The computer-implemented method of claim 11, further comprising:
receiving attenuation maps corresponding to the back-projected images; and
inputting the attenuation maps to the machine learning process and, based on inputting the attenuation maps to the machine learning process, generating the output data.

15. The computer-implemented method of claim 11, further comprising:
based on determining the machine learning process is trained:
receiving additional histo-projection data;
inputting the additional histo-projection data to the machine learning process and, based on inputting the additional histo-projection data to the machine learning process, generating additional output data;
generating an additional loss value based on the additional output data and additional ground truth data; and
determining the machine learning process is validated based on the additional loss value.

16. An apparatus comprising:
a memory storing instructions; and
at least one processor communicatively coupled the memory, wherein the at least one processor is configured to execute the instructions to:
receive image measurement data;
generate histo-projection data based on the image measurement data;
apply a trained machine learning process to the histo-projection data and, based on the application of the trained machine learning process to the histo-projection data, generate a reconstructed image; and
store the reconstructed image in a data repository.

17. The apparatus of claim 16, wherein, to apply the trained machine learning process to the histo-projection data, the at least one processor is configured to execute the instructions to:
generate features based on the histo-projection data; and
input the features to a trained machine learning model.

18. The apparatus of claim 16, wherein the at least one processor is configured to execute the instructions to:
receive an attenuation map; and
apply the trained machine learning process to the attenuation map and, based on the application of the trained machine learning process to the attenuation map, generate the reconstructed image.

19. The apparatus of claim 16, wherein the at least one processor is configured to execute the instructions to:
apply an attenuation correction process to the histo-projection data to correct for attenuation; and
apply the trained machine learning process to the attenuation corrected histo-projection data.

20. The apparatus of claim 16, wherein the trained machine learning process is trained with histo-projection data and corresponding ground truth data characterizing reconstructed images.
